(19) United States
(12) Patent Application Publication
(10) Pub. No.: US 2016/0361419 A1
(43) Pub. Date: Dec. 15, 2016

(54) METHOD OF STABILIZING COMPOUNDS IN WATER, WATER COMPOSITONS THEREBY, AND ARTICLES CONTAINGING SAID WATER COMPOSITIONS

(71) Applicant: Robinson B. Gourley, Sarasota, FL (US)

(72) Inventor: Robinson B. Gourley, Sarasota, FL (US)

(21) Appl. No.: 15/161,853

(22) Filed: May 23, 2016

Related U.S. Application Data
(60) Provisional application No. 62/173,525, filed on Jun. 10, 2015.

(51) Int. Cl. A61K 47/02; A61L 15/44; A61K 33/40; A61L 15/18; C01B 15/037; C01B 11/02

(52) U.S. Cl. CPC A61K 47/02; C01B 15/037; C01B 11/022; A61K 33/40; A61L 15/18; A61L 15/44; A61L 2300/404

(57) ABSTRACT

Peroxides and chlorine dioxide (compounds) can be stabilized for long periods of time (years) by combining the compounds with water that has been infused with SG gas. Such stabilized materials can be used to infuse soft, solid substrates that can be used as sterile wipes, wound dressings, or the like.

This application is a utility application claiming priority from U.S. Provisional application having Ser. No. 62/173,525, filed Jun. 10, 2015.

### BACKGROUND OF THE INVENTION

Hydrogen peroxide (H₂O₂) in a water solution has long been recognized as a generally safe antimicrobial agent. However, hydrogen peroxide is thermodynamically unstable and decomposes to form water and oxygen. Consequently, the effectiveness of using hydrogen peroxide as a disinfectant has mixed results and causes hospitals and other medical institutions to resort to chlorine-based bleaches which have environmental and health safety concerns.

Hydrogen peroxide has historically been used in disinfecting topical wounds until researchers proved that it destroyed skin cells, slowed healing and led to scarring. Only very low concentrations of hydrogen peroxide in water solution should be applied topically to wounds but again the stability of the hydrogen peroxide is a concern as well as the residue left afterwards on the wound by surfactants and other chemicals used by chemists in trying to stabilize the very low concentrations of hydrogen peroxide in water.

Hydrogen peroxide is thermodynamically unstable and decomposes to form water and oxygen. Hydrogen peroxide is seen as an environmentally safe alternative to chlorine-based bleaches, as it degrades to form oxygen and water. It can be used for the disinfection of various surfaces and is generally recognized as safe as an antimicrobial agent by the U.S. Food and Drug Administration (FDA). However studies have found that when diluted with regular water, it only retains its efficacy for sterilization for a short amount of time. Hydrogen peroxide decomposes to a low level of residual activity within hours or a couple of days, and proves to be ineffective in certain cases and hospitals and other medical institutions are now being advised to use chlorine-based bleaches for disinfection.

Hydrogen peroxide demonstrates broad-spectrum efficacy against viruses, bacteria, yeasts, and bacterial spores and longer contact times are required for sporicidal activity.

Historically hydrogen peroxide has been used for disinfecting wounds, partly because of its low cost and prompt availability compared to other antiseptics, only a very low concentration of H₂O₂ can induce healing, and only if not repeatedly applied. Surgical use can lead to gas embolism formation. Despite this it is still used for wound treatment in many developing countries. It is absorbed by skin upon contact and creates a local capillary embolism that appears as a temporary whitening of the skin.

Owing to the instability of hydrogen peroxide in water, it has become traditional for manufacturer's to put low concentrations of hydrogen peroxide (3%) in opaque bottles, or brown colored glass bottles, to preserve the stability. However, this only works for short periods of time.

### THE INVENTION

What has now been discovered is that peroxides and chlorine dioxide (compounds) can be stabilized for long periods of time (years) by combining the compounds with water that has been infused with SG gas.

Thus, this invention, in one embodiment, deals with a method of stabilizing a compound in water. The compounds are peroxides and chlorine dioxide. The method comprises combining a compound with water infused with SG gas.

SG gas is a novel material produced by the methods set forth in U.S. Patent Publication 2010/0209360 A1, published Aug. 19, 2010 in the name of the inventor herein which is incorporated herein for what it teaches with regard to the manufacture of SG gas and SG gas infused water.

Another embodiment of this invention deals with a composition of matter comprising water infused with SG gas and a compound selected from the group consisting of peroxides and chlorine dioxide.

Representative peroxides are, for example, hydrogen peroxide and benzoyl peroxide

A further embodiment of this invention is an article, the article comprising a soft, solid substrate containing a exposition as set forth Supra. Preferred soft, solid substrates are cotton and fabric gauze.

### DETAILED DESCRIPTION OF THE INVENTION

Thus, this invention deals with water that has been infused with SG gas and, hydrogen peroxide, in a concentration ranging between 0.1 weights and 3 weight % that is used to moisten a soft, solid substrate prior to sealing it in an individual packet. Such soft, solid materials can be used, for example, as sterile wipes, for cleaning of topical wounds, and for removal of bandage adhesives from the skin, and the like.

In a first step of the method of preparing SG gas, an aqueous fluid is provided to a reaction zone. While various aqueous fluids, such as distilled water, tap water, or water taken from a river, stream, lake or the like may be used to generate electrical current at satisfactory levels, it is preferred to use an electrolyte solution for the aqueous fluid of standardized composition so that the conditions of the method can be better standardized for maximum yield of gas.

The aqueous fluid is provided to a reaction zone which is preferably closed off so to allow the reaction to occur under pressure. An alkali salt is preferably used as an electrolyte dissolved in distilled water. Preferred alkali salts are tripotassium phosphate, potassium hydroxide, lithium hydroxide and sodium hydroxide. The specific gravity of the alkali salt in the solution is above 1.0. Most preferably, potassium hydroxide is employed at a specific gravity from at least above 1.0 up to about 1.2. If another electrolyte is chosen other than potassium hydroxide a mole ratio must be calculated for that substance so that the maximum mole ratio represented by the specific gravity of 1.2 provided for potassium hydroxide will not be exceeded. These specific gravity values are as determined by a refractometer which provides readings that are temperature compensated. Most preferably, the electrolyte employed is tripotassium phosphate dissolved in distilled water at a concentration sufficient to form a solution having up to 1.2 specific gravity. A suitable refractometer is the Westover Model RHA-100, portable refractometer.

Aqueous fluid is contained in a receptacle which can be made out of a variety of materials including sheet steel, stainless steel, CV-PVC and epoxy resin fiberglass. The apparatus and internal devices need to be heat resistant and waterproof. The reaction zone is comprised of said aqueous fluid.

The aqueous fluid is placed in a reaction zone in the method of the invention. Overall, the method employs creation of a magnetic field in the aqueous fluid and periodic collapse of the magnetic field under conditions which do not provoke electrolysis of the aqueous fluid. Under these conditions, a single gas is generated and collected. This gas has desirable properties and is useful for applications.

In a first step of the method, a magnetic field is applied to the reaction zone. Preferably, the magnetic field is applied by providing a source of electric power to said reaction zone. An electric current in said reaction zone provides a magnetic field.

In a preferred embodiment, two metallic end plates having an inside surface and an outside surface, and having the capacity to conduct an electrical current are used in the reaction zone in opposing configuration. The inside of each end plate is partially submerged in the electrolyte solution. The metallic plates are preferably comprised of nickel alloy or stainless steel, but any metal can be used as long as such metal has the capacity to conduct an electric current and is preferably resistant to corrosion or erosion by alkali solutions. One of said metallic plates serves as a cathode and the other as an anode. The cathode and anode should be separated a sufficient distance so that a magnetic field forms when current is applied to the reaction zone. The distance between the plates should be less than ¼ inch and up to an inch in the method of the invention. This distance is independent of the volume of the aqueous fluid employed or size of the reaction zone.

There is a relationship between the concentration of electrolyte solution and the amperage which will exist in the aqueous fluid upon application of current thereto. The higher the specific gravity, the greater the amperage will result. This will also affect the strength of the magnetic field, and increase the temperature of the solution. Electrolysis (used industrially to produce hydrogen gas via the reaction 2H₂O(l)→2H₂(g)+O₂(g)) which is not desired in the method of the invention, could occur if the current is too high. In order for the magnetic field to be applied to the reaction zone, a power source (e.g., 110 volts DC) is applied respectively to the anode and to the cathode.

An appropriate power source that may be used in the method of this invention is 110 volt alternating current which has been converted to direct current using a rectifying process (e.g., a diode bridge device). Any standard power or voltage source may be used as long as it is rectified to direct current. When an electric current is applied to the reaction zone, a magnetic field is created in the reaction zone, which periodically collapses and causes the conversion of the water in the aqueous fluid into gas. Cyclic pulsation will be present in current even after alternating current is converted to direct current (for example a 60 cycle pulsation from household current) unless a smoothing circuit has been incorporated. This resulting cyclic pulsation is employable in the invention to periodically collapse the magnetic field, however using an auxiliary pulsing unit is preferably used in the method of the invention so that better regulation of pulsing may be employed. Any means for causing the electric current provided to the reaction zone to pulse at a frequency of 15 to 20 kilohertz decreases the wattage needed to create gas by approximately a factor of 10. The amount of energy needed to generate one (1) liter of gas is 0.0028 kilowatt-hour and with a pulsing device associated with the reaction zone, the amount drops to 0.00028 kilowatt-hour or less to generate one (1) liter of gas.

As the pulsing occurs, the stationary magnetic field alternatively collapses and is reinstated. It has been found that a reaction occurs in the electrolyte solution between the two end plates upon collapse of the magnetic field, which results in a release of a generated gas, i.e. named herein as SG gas. Some of the same gas will be pulled toward the individual plates and released as part of the generated gas.

The SG gas generated by this method is then used to infuse water, the amount of infusion depending on the desired amount for a particular end use. Generally, the water is saturated with the gas.

This gas infused water is then treated with the desired peroxide or ClO₂ at the level desired, by merely mixing the peroxide with the water. The water is then used to moisten soft, solid substrates such as cotton or gauze and then the moistened substrates are individually packaged in packets for later use.

It is a unique property of the SG gas infused water, that it stabilizes any peroxide or ClO₂ for long periods of time that enables one to treat soft, solid substrates that will last a long time.

### EXAMPLES

### Example 1

Peroxide Test Strips supplied by Indigo® Instruments, Part #33815-P100 (referred as “Peroxide Test Strips” or “Test Strip” herein) were used to determine the amount of hydrogen peroxide in either distilled water or in water infused with SG Gas (referred as “SG Gas-infused Water” or “Ultra-pure Polarized Water” herein). Test Strips are capable of measuring peroxide concentrations between 1 and 100 ppm (parts per million). A Peroxide Test Strip was first submerged into 3% water solution of hydrogen peroxide obtained from a drugstore for home use. The Test Strip showed a reading of 100 ppm for peroxide as expected.

A Peroxide Test Strip was submerged into a clear PET-1 bottle (0.5 liter) of distilled water produced by Crystal Springs and another clear PET-1 bottle (0.5 liter) of SG Gas-infused Water manufactured by AquaNew, LLC, Sarasota, Fla., respectively. The Water produced by AquaNew is highly purified resulting from at least six (6) stages of treatment to obtain on or about 0.3 ppm of Total Dissolved Solids (pharmaceutical-grade water). No color change of each Test Strip was observed which indicates that peroxides were not present in either water sample even at the level of 1 ppm. Ten (10) drops of peroxide were added into each sample bottle and a Test strip was dipped into each bottle and each bottle was immediately recapped. The contents of each bottle measured a concentration of 50 ppm of peroxide. Both bottles with closures were left on a shelf in open light conditions under climate controlled conditions with no direct sunlight.

After two weeks, a Test Strip was dipped into each bottle and the bottle was immediately recapped. The distilled water contents showed a decline from 50 to 30 ppm of peroxide. The SG Gas-infused water remained stable at 50 ppm. The two bottles with closures were placed back onto the shelf.

After four (4) months, another Test Strip was dipped into each bottle and the bottles were immediately recapped. The distilled water contents showed the lower 30 ppm measurement while the SG Gas-infused water held the peroxide concentration stable at 50 ppm.

While the mechanism of peroxide stability is not completely understood, SG Gas restructures highly purified water to maintain a neutral or lower pH and a relatively high Oxidation Reduction Potential (ORP) above +400. Initially at the introduction of the Gas into the water, a negative ORP is measured which flips to the positive ORP within 24 hours of gassing the water. The theory is that the restructured water makes the hydrogen more accessible.

It appears the SG Gas-infused Water has a low capacitance for intercellular communication, which alleviates pain and inflammation, and promotes healing on its own without the addition of any low concentration of hydrogen peroxide. The SG Gas-infused Water has become a new effective and economical antibacterial and antifungal preparation in providing minimal infection potential, enhanced wound recovery and little to no aide effects to wound trauma.

### Example 2

Continuous experiments on the relative stabilization of hydrogen peroxide with different aqueous carriers, that is, types of water, including SG gas-infused water, were carried out.

Very low concentrations of hydrogen peroxide (1.2 weight %) was added to the various aqueous carriers and measured for percent absorption by potassium permanganate titration.

The samples were tested on day 1, 20, 27 and 34. The tests were accelerated in aging when the temperature of each water sample was maintained at 58° C., approximating close to two years of stabilization.

The SG gas-infused water samples prevent degradation of hydrogen peroxide at a temperature of 58° C. and maintain close to 100% absorption, that is, 1.2% concentration throughout the 34 days of testing. In comparison, hydrogen peroxide degraded in tap water at a temperature of 58° C. from 1.2% to 0.4% concentration on day 20 and below 0.2% concentration on day 34.

What is claimed is:

1. A method of stabilizing a compound in water, said compound selected from the group consisting of:
i. peroxides, and,
ii. chlorine dioxide,
said method comprising combining said compound with water infused with SG gas.

2. A composition of matter comprising:
a. water infused with SG gas and,
a compound selected from tie group consisting of
i. peroxides, and,
ii. chlorine dioxide.

3. A composition as claimed in claim 2 wherein the peroxide is hydrogen peroxide.

4. A composition as claimed in claim 2 wherein the peroxide is benzoyl peroxide.

5. An article, said article comprising a soft, solid substrate containing a composition as claimed in claim 2.

6. An article as claimed in claim 5 wherein the soft, solid substrate is cotton.

7. An article as claimed in claim 5 wherein the soft, solid substrate is fabric gauze.
